(19) United States
(12) Patent Application Publication
(10) Pub. No.: US 2013/0189218 A1
(43) Pub. Date: Jul. 25, 2013

(54) PHARMACEUTICAL FORMULATIONS FOR FUMAGILLIN DERIVATIVE-PHF CONJUGATES

(71) Applicants: Laura Akullian, Lunenburg, MA (US); Gui Liu, Lexington, MA (US); Timothy B. Lowinger, Carlisle, MA (US); Dennis McGillicuddy, Reading, MA (US); Cheri Stevenson, Haverhill, MA (US); John Van Duzer, Georgetown, MA (US); Mao Yin, Needham, MA (US); Aleksandr Yurkovetskiy, Littleton, MA (US)

(72) Inventors: Laura Akullian, Lunenburg, MA (US); Gui Liu, Lexington, MA (US); Timothy B. Lowinger, Carlisle, MA (US); Dennis McGillicuddy, Reading, MA (US); Cheri Stevenson, Haverhill, MA (US); John Van Duzer, Georgetown, MA (US); Mao Yin, Needham, MA (US); Aleksandr Yurkovetskiy, Littleton, MA (US)

(73) Assignee: Mersana Therapeutics, Inc., Cambridge, MA (US)

(21) Appl. No.: 13/725,900

(22) Filed: Dec. 21, 2012

Related U.S. Application Data
(60) Provisional application No. 61/639,654, filed on Apr. 27, 2012.
(60) Provisional application No. 61/580,016, filed on Dec. 23, 2011.

(51) Int. Cl. A61K 47/48

(52) U.S. Cl. CPC A61K 47/48192
USPC 424/78.17; 525/438

(57) ABSTRACT

The invention described herein provides a mixture comprising polymer molecules or salts thereof, wherein a polymer molecule in the mixture comprises covalently bound subunits L, K, and M wherein the average molecular weight of the polymer molecules in the mixture is about 50 kDa to about 200 kDa, wherein the mole percentage of subunit M, K and L, relative to the total amount of subunits in the mixture, is about 90.5 to about 96 mol %, about 2.8 to about 7.3 mol %, and about 1.2 to about 2.2 mol %, respectively.

This application claims benefit of U.S. Provisional Application Nos. 61/639,654, filed Apr. 27, 2012 and 61/580,016, filed Dec. 23, 2011, the contents of each of which are hereby incorporated by reference in their entireties.

Throughout this application various publications are referenced. The disclosures of these documents in their entireties are hereby incorporated by reference into this application in order to more fully describe the state of the art to which this invention pertains.

### BACKGROUND OF THE INVENTION

Fumagillin is a known natural compound which has been used as an antimicrobial and antiprotozoal. Its physicochemical properties and method of production are well known (U.S. Pat. No. 2,803,586 and Proc. Nat. Acad. Sci. USA (1962) 48:733-735). Fumagillin and certain types of fumagillin analogs have also been reported to exhibit anti-angiogenic activity. However, the use of such inhibitors (e.g., TNP-470) may be limited by their rapid metabolic degradation, erratic blood levels, and by dose-limiting central nervous system (CNS) side effects. Additionally, these molecules have physical and chemical properties that make them undesirable as therapeutics, for example, low water solubility, very short half-life values and unacceptable neurotoxic side-effects.

These problems can be overcome or significantly diminished while maintaining their biological activities by linking fumagillin derivatives to poly(1-hydroxymethylethylene hydroxymethyl-formal) (PHF). PHF molecules, such as Fleximer®, Mersana Therapeutics, Inc. (Cambridge, Mass.) are described in U.S. Pat. No. 5,811,510, U.S. Pat. No. 5,863,990, U.S. Pat. No. 5,958,398 and U.S. Patent Application Publication Number US/2009/0148396 A1.

PHF contains acetals which are known to degrade at low pH (Papisov, et al., Biomacromolecules (2005) 6: 2659-70). Furthermore, fumagillin derivatives are attached to PHF via one or more labile bonds, such as, for example, ester and amide linkages which tend to hydrolyze at high pH. Different fumagillin derivative-PHF conjugates therefore have components that; may be individually destabilized at basic pH and acidic pH. These characteristics vary for different conjugates but often make handling difficult, particularly handling of aqueous solutions of certain fumagillin derivative-PHF conjugates.

Disclosed herein is a fumagillin derivative-PHF conjugate which has specific needs for a stable formulation. In addition, the disclosed fumagillin derivative-PHF conjugate has specific issues and needs with respect to its ability to form injectable lyophilized powder that must be reconstituted in Sterile Water for Injection, USP or Sodium. Chloride Injection, USP for infusion. Thus, there is also a need for specific formulations of the fumagillin derivative-PHF conjugate that can be rapidly dissolved to minimize preparation time in hospital or clinic pharmacy.

### SUMMARY OF THE INVENTION

The invention described herein provides a mixture comprising polymer molecules or salts thereof, wherein a polymer molecule in the mixture comprises covalently bound subunits L, K, and H represented as:

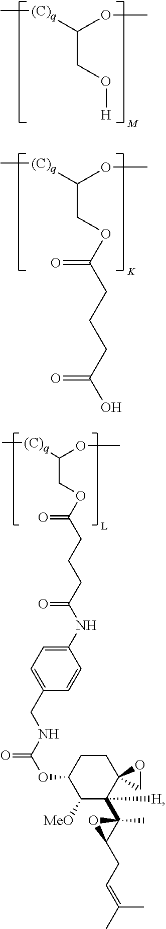

wherein q=0 or 1,
wherein every subunit which is bound to a subunit where q is 1 is a subunit where q is 0 and every subunit which is bound to a subunit where q is 0 is a subunit where q is 1 such that subunits where q is 0 and subunits where q is 1 alternate in the polymer molecule,
wherein the average molecular weight, of the polymer molecules in the mixture is about to 50 kDa to about 200 kDa,
wherein the mole percentage of subunit M, relative to the total amount of subunits; in the mixture, is about 90.5 to about 96 mol %,
wherein the mole percentage of subunit K, relative to the total amount, of subunit s in the mixture, is about 2.8 to about 7.3 mol %, and
wherein the mole percentage of subunit L, relative to the total amount of subunits in the mixture, is about 1.2 to about 2.2 mol %.

The invention, also provides a mixture comprising polymer molecules or salts thereof, wherein a polymer molecule in the mixture comprises a poly(1-hydroxymethylethylone hydroxymethyl-formal) backbone having covalently bound thereto through a carboxyl group glutaric acid and Compound D of the formula:

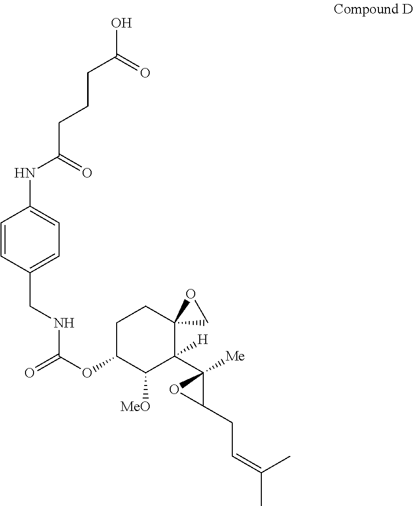

wherein the average molecular weight of the polymer molecules in the mixture is about 50 kDa to about 200 kDa,
wherein the mole percentage of glutaric acid covalently bound to the mixture of polymer molecules, relative to the total amount of subunits in the mixture, is about 2.8 to about 7.3 mol %, and
wherein the mole percentage of Compound D covalently bound to the mixture of polymer molecules, relative to the total amount of subunits in the mixture, is about 1.2 to about 2.2 mol %.

The invention also provides a process for producing a mixture comprising polymer molecules, wherein a polymer molecule in the mixture comprises covalently bound subunits L, K, and M represented as:

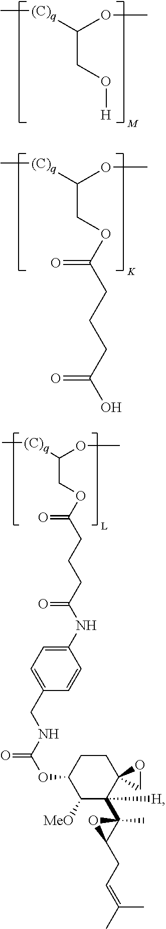

wherein q=0 or 1,
wherein every subunit which is bound to a subunit where q is 1 is a subunit where q is 0 and every subunit which is bound to a subunit where q is 0 is a subunit where q is 1 such that subunits where q is 0 and subunits where q is 1 alternate in the polymer molecule,
wherein the average molecular weight of: the polymer molecules in the mixture is about 50 kDa to about 200 kDa,
wherein the mole percentage of subunit M, relative to the total amount of subunits in the mixture, is about 90.5 to about 96 mol %,
wherein the mole percentage of subunit K, relative to the total amount of subunits in the mixture, is about 2.8 to about 7.3 mol %, and
wherein the mole percentage of subunit L, relative to the total amount of subunits in the mixture, is about 1.2 to about 2.2 mol %, the process comprising,
a) obtaining a mixture of PHF-GA molecules having at least 3 mole percentage of subunit K, relative to the total amount of subunits in the mixture of PHF-GA molecules,
b) reacting Compound B with the mixture of PHF-GA molecules,
   thereby producing the mixture comprising the polymer molecules.

The invention also provides a method of treating cancer, comprising administering to a subject in need thereof a mixture of any one of the embodiments of the invention, or a pharmaceutical formulation of any of the embodiments of the invention in an amount effective to treat the cancer.

The invention also provides the use of a mixture or a pharmaceutical formulation of one or more embodiments of the invention in the treatment of cancer.

The invention also provides the use of a mixture or a pharmaceutical formal at ion of one or more embodiments of the invention in the manufacture of a medicament for treatment of cancer.

The invention also provides a method of treating an angiogenic disease, comprising administering to a subject in need thereof a mixture of any one of the embodiments of the invention, or a pharmaceutical formulation of any of the embodiments of the invention in an amount effective to treat the angiogenic disease.

The invention also provides the use of a mixture or a pharmaceutical formulation of one or more embodiments of the invention in the treatment of an angiogenic disease.

The invention also provides the use of a mixture or a pharmaceutical formulation of one or more embodiments of the invention in the manufacture of a medicament for treatment of an angiogenic disease.

The invention also provides a method of delivering Compound D of the formula:

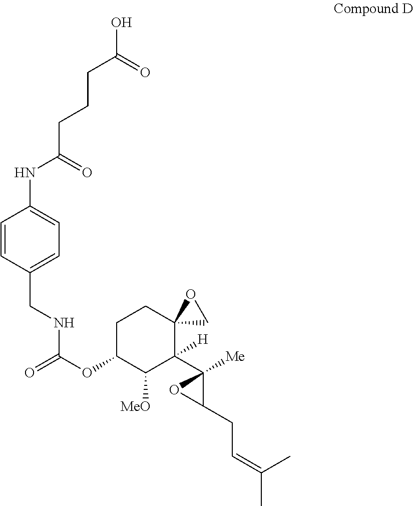

to a subject comprising administering to the subject a mixture of or a pharmaceutical formulation of one or more embodiments of the invention.

### BRIEF DESCRIPTION OF THE FIGURES

FIG. 1. High Performance Size Exclusion Chromatography Traces of Different Compound D Loading Conjugates (HPSEC). Composition C batches with different Compound D loading were analyzed by HPSEC. Lower Compound D loading resulted in a single peak while 2 peaks were observed for higher Compound B loading samples.

### DETAILED DESCRIPTION OF THE INVENTION

Described herein are novel pharmaceutical formulations for fumagillin derivative-PHF conjugates.

The invention described herein provides a mixture comprising polymer molecules or salts thereof, wherein a polymer molecule in the mixture comprises covalently bound subunits L, K, and M represented as:

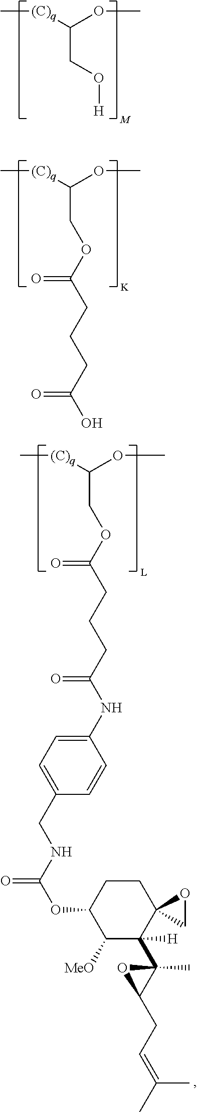

wherein q=0 or 1,
wherein every subunit which is bound to a subunit where q is 1 is a subunit where q is 0 and every subunit which is bound to a subunit where q is 0 is a subunit where q is 1 such that subunits where q is 0 and subunits where q is 1 alternate in the polymer molecule,
wherein the average molecular weight of the polymer molecules in the mixture is about 50 kDa to about 200 kDa,
wherein the mole percentage of subunit M, relative to the total amount of subunits in the mixture, is about 90.5 to about 96 mol %,
wherein the mole percentage of subunit K, relative to the total amount of subunits in the mixture, is about 2.8 to about 7.3 mol %, and
wherein the mole percentage of subunit L, relative to the total amount of subunits in the mixture, is about 1.2 to about 2.2 mol %.

In one or more embodiments the mole percentage of subunit M, relative to the total amount of subunits in the mixture, is about 91.5 to about 96 mol %.

In one or more embodiments the mole percentage of subunit M, relative to the total amount of subunits in the mixture, is about 93.5 to about 95 mol %.

In one or more embodiments the mole percentage of subunit K, relative to the total amount of subunits in the mixture, is about 3.0 to about 6.0 mol %.

In one or more embodiments the mole percentage of subunit K, relative to the total amount of subunits in the mixture, is about 2.8 to about 4.9 mol %.

In one or more embodiments the mole percentage of subunit L relative to the total amount of subunits in the mixture is about 1.2% to about 2.2%, about 1.4% to about 2.2%, about 1.6% to about 2.2%, about 1.4% to about 2.1%, about 1.5% to about 2.0%, about 1.6% to about 2.0%, or about 1.7% to about 1.9%, or about 1.75%, or about 1.80%.

In one or more embodiments the mole percentage of subunit L, relative to the total amount of subunits in the mixture, is about 1.6 to about 2.2 mol %. The invention also provides a mixture comprising polymer molecules or salts thereof, wherein a polymer molecule in the mixture comprises a poly(1-hydroxymethylethylene hydroxymethyl-formal) backbone having covalently bound thereto glutaric acid and Compound D of the formula:

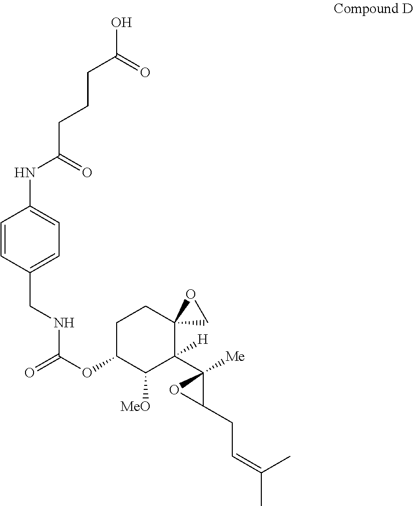

wherein the average molecular weight of the polymer molecules in the mixture is about 50 kDa to about 200 kDa,
wherein the mole percentage of glutaric acid covalently bound to the mixture of polymer molecules, relative to the total amount of subunits in the mixture, is about 2.8 to about 7.3 mol %, and
wherein the mole percentage of Compound D covalently bound to the mixture of polymer molecules, relative to the total amount of subunits in the mixture, is about 1.2 to about 2.2 mol %.

In one or more embodiments the mole percentage of glutaric acid covalently bound to the mixture of polymer molecules, relative to the total amount of subunits in the mixture, is about 3.0 to about 6.0 mol %.

In one or more embodiments the mole percentage of glutaric acid covalently bound to the mixture of polymer molecules, relative to the total, amount of subunits in the mixture, is about 2.8 to about 4.9 mol %.

In one or more embodiments the mole percentage of Compound D covalently bound to the mixture of polymer molecules, relative to the total amount of subunits in the mixture is about 1.2% to about 2.2%, about 1.4% to about 2.2%, about 1.6% to about 2.2%, about 1.4% to about 2.1%, about 1. %% to about 2.0%, about 1.6% to about 2.0%, or about 1.7% to about 1.9%, or about 1.75%, or about 1.80%.

In one or more embodiments the mole percentage of Compound D covalently bound to the mixture of polymer molecules, relative to the total amount of subunits in the mixture, is about 1.6 to about 2.2 mol %.

In one or more embodiments the average molecular weight, of the polymer molecules in the mixture is about 70 kDa,

In one or more embodiments the peak molecular weight of the mixture of polymer molecules is less than 100 kDa.

In one or more embodiments the molecular weight distribution of the mixture of polymer molecules has a single peak.

In one or more embodiments the peak molecular weight of the mixture of polymer molecules is less than 70 kDa.

In one or more embodiments the peak molecular weight of the mixture of polymer molecules is about 40 kDa to about 60 kDa.

In one or more embodiments D₁₀ of the molecular weight distribution of the mixture of polymer molecules is less than or equal to 50 kDa.

In one or more embodiments of the molecular weight distribution of the mixture of polymer molecules is less than or equal to 200 kDa.

In one or more embodiments D₉₀ of the molecular weight distribution of the mixture of polymer molecules is less than or equal to 300 kDa.

In one or more embodiments the mixture further comprises one or more impurities, wherein the one or more impurities are present in an amount of less than 5% by weight.

In one or more embodiments impurities are present in an amount of about 1% to about 5% by weight.

In one or more embodiments the salt is a pharmaceutical acceptable salt.

The invention also provides a pharmaceutical formulation comprising a mixture of any of the embodiments of the invention.

In one or more embodiments the pharmaceutical formulation further comprises one or more buffers.

In one or more embodiments the one or more buffers are selected from the group consisting of sodium citrate, citric acid, ascorbate, succinate, lactate, boric acid, borax, disodium hydrogen phosphate, acetic acid, formic acid, glycine, bicarbonate, tartaric acid, Tris-glycine, Tris-NaCl, Tris-EDTA, Tris-borate-EDTA, TAE-buffer, Tris-buffered saline, HEPES, MOPS, PIPES, MES, and PBS.

In one or more embodiments the selected buffers are sodium citrate and citric acid.

In one or more embodiments the formulation is buffered to a pH of about 5 to about 6.

In one or more embodiments the formulation is buffered to about pH 5.5.

In one or more embodiments the pharmaceutical formulation further comprises one or more stabilizing agents.

In one or pore embodiments the one or more stabilizing agents are selected from the group consisting of mannitol, sorbitol, maltose, trehalose, polyvinyl pyrrolidone sucrose, lactose, fructose, raffinose, hydroxylpropyl-β-cyclorlextrin glucose, xylitol, and lactitol.

In one or more embodiments the stabilizing agent is mannitol.

In one or more embodiments mannitol is present in the pharmaceutical formulation in an amount of about 35% to about 50% by weight.

In one or more embodiments mannitol is present in the pharmaceutical formulation in an amount of about 42% by weight.

In one or more embodiments the pharmaceutical formulation further comprises one or more surfactants.

In one or more embodiments the one or more surfactants are selected from the group consisting of Polysorbate 80, Poloxamer 407, Polysorbate 20, Poloxamer 188, Solutol HS 15, Tween 80, sodium lauryl sulphate, ether sulphates, sulphated oils, cetrimide BP, benzalkonium chloride, lecithin, cetromacrogel 1000 BPC, and alkali metal soaps of the formula RCOOX where R=C₁₀-C₂₀ alkyl group, and X=sodium, potassium, or ammonium.

In one or more embodiments the formulation is a stable aqueous solution.

In one or more embodiments the formulation is a stable lyophilized formulation.

In one or more embodiments the lyophilized formulation contains about 8.4% sodium citrate by weight.

In one or more embodiments the lyophilized formulation contains about 1.2% citric acid by weight.

In one or more embodiments the lyophilized formulation contains less than or equal to about 4% water by weight.

In one or more embodiments the lyophilized formulation is suitable for intravenous administration after reconstitution with a reconstitution agent.

In one or more embodiments the reconstitution agent is 0.9% Sodium Chloride Injection, USP.

In one or more embodiments the reconstitution agent is sterile water for injection, USP.

In one or more embodiments the pharmaceutical formulation further comprises one or more preservatives.

In one or more embodiments the one or more preservatives are selected from the group consisting of benzyl alcohol, sodium benzoate acid, sodium nitrate, sulphur dioxide, sodium sorbate and potassium sorbate.

The invention also provides a process for producing a mixture comprising polymer molecules, wherein a polymer molecule in the mixture comprises covalently bound subunits L, K, and M represented as:

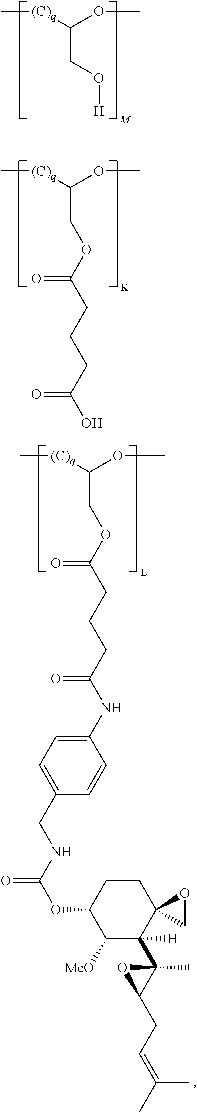

wherein q=0 or 1,
wherein every subunit which is bound to a subunit where q is 1 is a subunit where q is 0 and every subunit which is bound to a subunit where q is 0 is a subunit where q is 1 such that subunits where q is 0 and subunits where q is 1 alternate in the polymer molecule.
wherein the average molecular weight of the polymer molecules in the mixture is about 50 kDa to about 200 kDa,
wherein the mole percentage of subunit M, relative to the total amount of subunits in the mixture, is about 90.5 to about 96 mol %,
wherein the mole percentage of subunit K, relative to the total amount of subunits in the mixture, is about 2.8 to about 7.3 mol %, and
wherein the mole percentage of subunit L, relative to the total amount of subunits in the mixture, is about 1.2 to about 2.2 mmol %, the process comprising,
a) obtaining a mixture of PHF-GA molecules having at least 3 mole percentage of subunit K, relative to the total amount of subunits in the mixture of PHF-GA molecules,
b) reacting Compound B with the mixture of PHF-GA molecules,
   thereby producing the mixture comprising the polymer molecules.

In one or more embodiments the mole percentage of subunit M, relative to the total amount of subunits in the mixture, is about 91.5 to about 96 mol %.

In one or more embodiments the mole percentage of subunit M, relative to the total amount of subunits in the mixture, is about 93.5 to about 95 mol %.

In one or more embodiments the mole percentage of subunit K, relative to the total amount of subunits in the mixture, is about 3.0 to about 6.0 mol %.

In one or more embodiments the mole percentage of subunit K, relative to the total amount of subunits in the mixture, is about 2.8 to about 4.9 mol %.

In one or more embodiments the mole percentage of subunit L relative to the total amount of subunits in the mixture is about 1.2% to about 2.2%, about 1.4% to about 2.2%, about 1.6% to about 2.2%, about 1.4% to about 2.1%, about 1.5% to about 2.0%, about 1.6% to about: 2.0%, or about 1.7% to about 1.9%, or about 1.75%, or about 1.80%.

In one or more embodiments the mole percentage of subunit L, relative to the total amount of subunits in the mixture, is about 1.6 to about 2.2 mol %.

In one or more embodiments in step a) the mixture of PHF-GA molecules has about 4 mole percentage to about 6 mole percentage of subunit K, relative to the total amount of subunits in the mixture of PHF-GA molecules.

In one or more embodiments in step a) the Mol % of subunit K in the PHF-GA is about 3% to about 9.5%, about 4% to about 8%, or about 5% to about 6.5%, or about 5.6%, or about 6.9%. In one or more embodiments the process further comprises in step b), maintaining the pH of the reaction at about pH 4 to about pH 6.

In one or more embodiments the pH is maintained at about pH 5.5.

In one or more embodiments the process further comprises purifying the product by diafiltration using a filter.

In one or more embodiments the filter has a nominal MWCO of 10 kDa.

The invention also provides a method of treating cancer, comprising administering to a subject in need thereof a mixture of any one of the embodiments of the invention, or a pharmaceutical formulation of any of the embodiments of the invention in an amount effective to treat the cancer.

The invention also provides the use of a mixture or a pharmaceutical formulation of one or more embodiments of the invention in the treatment of cancer.

The invention also provides the use of a mixture or a pharmaceutical formulation of one or more embodiments of the invention in the manufacture of a medicament for treatment of cancer.

In one or more embodiments the cancer is anal, astrocytoma, leukemia, lymphoma, head and neck, liver, testicular, cervical, sarcoma, hemangioma, esophageal, eye, laryngeal, mouth, mesothelioma, skin, myeloma, oral, rectal, throat, bladder, breast, uterus, ovary, prostate, lung, colon, pancreas, renal or gastric.

The invention also provides a method of treating an angiogenic disease, comprising administering to a subject in need thereof a mixture of any one of the embodiments of the invention, or a pharmaceutical formulation of any of the embodiments of the invention in an amount effective to treat the angiogenic disease.

The invention also provides the use of a mixture or a pharmaceutical formulation of one or more embodiments of the invention in the treatment of an angiogenic disease.

The invention also provides the use of a mixture or a pharmaceutical formulation of one or more embodiments of the invention in the manufacture of a medicament for treatment of an angiogenic disease.

The invention also provides a method of delivering Compound o of the formula:

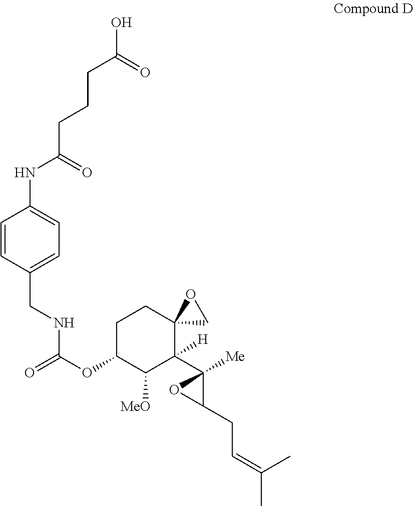

to a subject comprising administering to the subject a mixture of or a pharmaceutical formulation of one or more embodiments of the invention.

U.S. Patent Application Publication No. US 2009-0148396 A1 describes biocompatible biodegradable fumagillin analog conjugates and is incorporated herein by reference in its entirety.

For the foregoing embodiments, each embodiment disclosed herein is contemplated as being applicable to each of the other disclosed embodiments. Thus, all combinations of the various elements described herein are within the scope of the invention.

### DEFINITIONS

As used herein, and unless otherwise stated, each of the following terms shall have the definition set forth below.

A “subject” may be, but is not limited to, humans as well as non-human animals, at any stage of development, including, for example, mammals, birds, reptiles, amphibians, fish, worms and single cells. Cell cultures and live tissue samples are considered to be pluralities of animals. Preferably, the non-human animal is a mammal (e.g., a rodent, a mouse, a rat, a rabbit, a monkey, a dog, a cat, a primate, or a pig). An animal may be a transgenic animal or a human clone. The term “subject” encompasses animals.

The term “pharmaceutically acceptable salts” include, e.g., water-soluble and water-insoluble salts, and include salts of pharmaceutically acceptable anions and salts of pharmaceutically acceptable cations. Salts of pharmaceutically acceptable anions include acetate, amsonate (4,4-diaminestilbene-2,2-disulfonate), benzenesulfonate, benzoate, bicarbonate, bisulfate, bitartrate, borate, bromide, butyrate, calcium edetate, carnsylate, carbonate, chloride, citrate, clavulariate, dihydrochloride, edetate, edisylate, estolate, esylate, fumarate, gluceptate, gluconate, glutamate, glutarate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsuifate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. Salts of pharmaceutically acceptable cations include ammonium, arganine, benethamine, benzathine, betaine, choline, deanol, diethanolamine, diethylamine, 2-(diethylamino)ethanol, epolamine, ethanolamine, ethylenediamine, 1H-imidizole, histidine, hydrabamine, lysine, morpholineethanol, N-methyl glucamine, meglumine, piperazine, procaine, triethanolamine, triethylamine, trolamine and tromethamine salts. Other salts of pharmaceutical cations include salts of metals including, but not limited to Zn+2, Fe+2, Mg+2, Ca+2, Al+3, Li+ and K+ salts.

As used herein, “administering” an agent may be performed using any of the various methods or delivery systems well known to those skilled in the art. The administering can be performed, for example, orally, parenterally, intraperitoneally, intravenously, intraartetially, transderatally, sublinqually, intramuscularly, rectally, transbuccally, intranasaiiy, liposomally, via inhalation, vaginally, inrraoccularly, via local delivery, subcutaneously, intraadiposally, intraarticularly, intrathecally, into a cerebral ventricle, intraventiculariy, intraturoocally, into cerebral parenchyma or intraparenchchymally.

As used herein, “administering” includes co-administering, either simultaneously with, prior to or after the administration of another therapeutic agent; the therapeutic agent includes, but is not limited to, anti-cancer agents, anti-angiogenesis agents, or anti-inflammatory agents. As used herein, “PHF-GA” means a mixture comprising polymer molecules or pharmaceutically acceptable salts thereof, wherein a polymer molecule in the mixture comprises a poly(1-hydroxymethylethylene hydroxymethy 1-formal) backbone having covalently bound thereto glutaric acid through a carboxyl group.

The following delivery systems, which employ a number of routinely used pharmaceutical carriers, may be used but are only representative of the many possible systems envisioned for administering compositions in accordance with the invention.

Injectable drug delivery systems include solutions, suspensions, gels, microspheres, nano-spheres/nano-particles and polymeric injectables, and can comprise excipients such as solubility-altering agents (e.g., ethanol, propylene glycol and sucrose) and polymers (e.g., PVP, polycaprylactones and PLGA's).

Other injectable drug delivery systems include solutions, suspensions and gels. Oral delivery systems include tablets and capsules. These can contain excipients such as binders and bulking agents (e.g., hydroxypropyl methylcellulose, polyvinyl pyrilodone, Copovidone, other cellulosic materials and starch), diluents (e.g., lactose and other sugars, isomalt, polyols (e.g., mannitol and sorbitol, starch, dicalcium phosphate and cellulosic materials), disintegrating agents (e.g., Crospovidone, starch polymers and cellulosic materials) and lubricating agents (e.g., stearates, sodium stearyl fumerate, glyceryl behenate and talc), colors and flavors.

Implantable systems include rods and discs, and can contain excipients such as polyvinyl pyrrolidone, PLGA and polycaprylactone.

Oral delivery systems include tablets and capsules. These can contain excipients such as binders and bulking agents (e.g., hydroxypropylmethylcellulose, polyvinyl pyrrolidone, Copovidone, other cellulosic materials and starch), diluents (e.g., lactose and other sugars, isomalt, polyols (e.g., mannitol and sorbitol), starch, dicaicium phosphate and cellulosic materials), disintegrating agents (e.g., Crospovidone, starch polymers and cellulosic materials) and lubricating agents (e.g., stearates, sodium stearyl fumerate, glyceryl behenate and talc), colors and flavors.

Transmucosal delivery systems include patches, tablets, suppositories, pessaries, gels and creams, and can contain excipients such as solubilizers and enhancers (e.g., propylene glycol, bile salts and amino acids) anionic and ionic surfactants (e.g., sorbitan esters, polysorbates and SDS) and other vehicles (e.g., polyethylene glycol, fatty acid esters and derivatives, and hydrophilic polymers such as hydroxypropylcellulose hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, anionic and ionic surfactants (e.g., sorbitan esters, polysorbates and SDS), permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parubens, vitamins E and C, and ascorbic acid), anti-caking agents, coating agents, and chelating agents (e.g., EDTA).

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

As used herein, an “amount” or “dose” of an agent measured in milligrams refers to the milligrams of agent present in a drug product, regardless of the form of the drug product.

As used herein, the term “therapeutically effective amount” or “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, the term “angiogenic disease” includes a disease, disorder, or condition characterized or caused by aberrant or unwanted, e.g., stimulated or suppressed, formation of blood vessels (angiogenesis). Aberrant or unwanted angiogenesis may either cause a particular disease directly or exacerbate an existing pathological condition. Examples of angiogenic diseases include cancer, e.g., carcinomas and sarcomas, where progressive growth is dependent upon the continuous induction of angiogenesis by these tumor cells; pediatric disorders, e.g., angiofibroma, and hemophiliac joints; blood vessel diseases such as hemangiomas, and capillary proliferation within atherosclerotic plaques; disorders associated with surgery, e.g., hypertrophic scars, wound granulation and vascular adhesions; autoimmune diseases such as rheumatoid, immune and degenerative arthritis, where new vessels in the joint may destroy articular cartilage; and sclerodermaocular disorders and ocular disorders, e.g. diabetic retinopathy, retinopathy of prematurity, corneal graft rejection, retrolental fibroplasia, neovascular glaucoma, rubeosis, retinal neovascularization due to macular degeneration, hypoxia, angiogenesis in the eye associated with infection or surgical intervention, ocular tumors and trachoma, and other abnormal neovascularization conditions of the eye, where neovascularization may lead to blindness; and disorders affecting the skin, e.g., psoriasis and pyogenic granuloma, obesity, where adipogenesis is associated with neovascularization, and activated adipocytes produce multiple pro-angiogenic factors which can stimulate neovascularization during fat mass expansion; and endometriosis, where the endometriotic lesion is supported by the growth of new blood vessels, and the endometrium of patients with endometriosis shows enhanced endothelial cell proliferation.

The term angiogenic disease also includes diseases characterized by excessive or abnormal stimulation of endothelial cells, including but not limited to intestinal adhesions, Crohn's disease, atherosclerosis, scleroderma, and hypertrophic scars, i.e., keloids; diseases that have angiogenesis as a pathologic consequence such as cat scratch disease (Rochele ninalia quintosa) and ulcers (Helicobacter pylori). In addition, the angiogenesis inhibitor compounds of the present invention are useful as birth control agents (by virtue of their ability to inhibit the angiogenesis dependent ovulation and establishment of the placenta) and may also be used to reduce bleeding by administration to a subject prior to surgery.

As used herein the molecular weight distribution of a mixture of polymers of the present invention is defined as the apparent molecular weights of all polymer molecules of a sample when assayed against known molecular weight standards as described in Methods herein below. Weight average molecular weight (Mw), number average molecular weight (Mn), peak molecular weight (Mp), D₁₀, D₅₀ and D₉₀ are all values used to describe the molecular weight distribution of a sample.

As used herein, D₁₀, D₅₀ and D₉₀ are defined respectively as the molecular weight corresponding to the 10^{th}, 50^{th} and 90^{th} percentile of a molecular weight distribution described by signal versus retention time as described in Methods herein below. Therefore for a given conjugate mixture batch 10% of the total mass of the conjugate mixture will have a molecular weight at or below D₁₀, 50% of the total mass will have a molecular weight at or below D₅₀ and 90% of the total mass will have a molecular weight at or below D₉₀.

The term “PHF” refers to poly(1-hydroxymethylethylene hydroxymethyl-formal). PHF can be derived from exhaustively oxidized dextran followed by reduction as described in U.S. Pat. No. 5,811,510, which is hereby incorporated by reference for its description of polyacetals, inter alia, at column 2, line 65 to column 8, line 55 and their synthesis at column 10, line 45 to column 11, line 14.

The poly(1-hydroxymethylethylene hydroxymethyl formal) polymers comprise unmodified monomer acetal units of Formula (I):

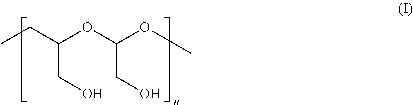

wherein n is the number of subunits of Formula (I) present in a polymer molecule of the mixture.

The PHF polymers can also be described as comprising subunits of Formula II:

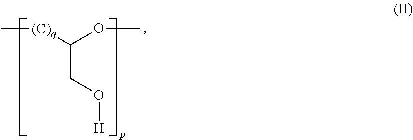

wherein p is the molar fraction of subunits of Formula (II) present in the polymer molecules of the mixture and wherein q=0 or 1 and wherein every subunit which is bound to a subunit where q is 1 is a subunit where q is 0 and every subunit which is bound to a subunit where q is 0 is a subunit where q is 1 such that subunits where q is 0 and subunits where q is 1 alternate in the polymer molecule, which gives the polymer arrangement of Formula I as shown below.

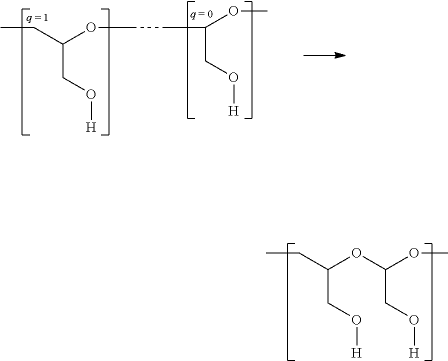

Accordingly, in one or more embodiments of the invention, polymer molecules in a mixture comprise a series of consecutively bound subunits, wherein the value of q for each successively bound subunit is 0, followed by 1, followed by 0, followed by 1, followed by 0, etc resulting in an alternating copolymer of glycerol and glycol aldehyde.

In one or more embodiments the overage molecular weight or the unmodified PHF is between about 0.5 and about 250 kDa. In a preferred embodiment the molecular weight is between about 1 and about 200 kDa (e.g., between about 5 and about 150 kDa, between about 10 and about 125 kDa, between about 20 and about 100 kDa, between about 49 kDa and about 77 kDa, or about 56 kDa, or about 70 kDa).

In one embodiment, the modified polymer backbone comprises subunits of Formula (III):

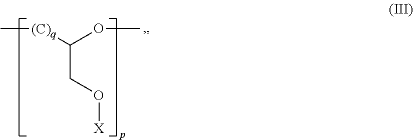

wherein X indicates an optional substituent for the hydroxyl group of the polymer backbone and wherein in each subunit, X is independently unsubstituted (X═H) or independently selected from a group consisting of one or more substituents and wherein p is molar fraction of subunits of Formula (III) present in the polymer molecules of the mixture.

The molar fraction p, of unmodified (X═H) subunits is the molar fraction available to promote biocompatibility, solubility and increase half-life. The molar fraction is based on the total number of subunits in the mixture of polymer molecules. The molar fraction p may be the minimal fraction of unmodified monomer subunits needed to provide biocompatibility, solubility, stability, or a particular half-life, or can be some larger fraction. The most desirable degree of cytotoxicity is substantially none, i.e., the modified polymer is substantially inert to the subject. However, as is understood by those of ordinary skill in the art, some degree of cytotoxicity can be tolerated depending on the severity of disease or symptom being treated, the efficacy of the treatment, the type and degree of immune response, and like considerations.

In a specific embodiment herein, in each subunit X, is independently selected from structures m, k and l:

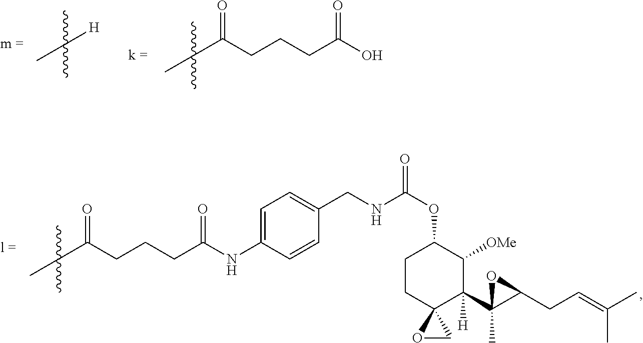

wherein the presence of m represents an unsubstituted conjugation site on the polymer sidechain, and wherein the presence of k represents conjugation of glutaric acid to the polymer sidechain, and wherein the presence of 1 represents conjugation of the shown compound to the polymer sidechain. Polymers of this embodiment can be represented as being composed of subunits M, K and L representing X=m, X=k and X=l respectively. In the present invention, when each of M, K, and L are present in a mixture of polymer molecules in specific Mol % ratios, wherein q=0 or 1 and wherein every subunit which is bound to a subunit where q is 1 is a subunit where q is 0 and every subunit which is bound to a subunit where q is 0 is a subunit where q is 1 such that subunits where q is 0 and subunits where q is 1 alternate in the polymer molecule such that the atoms forming the continuous backbone of a polymer molecule are in the same conformation as corresponding atoms that form the continuous backbone of Dextran as shown in Example 1, are known as Composition C.

In one or more embodiments polymer molecules in the mixture comprise a series of consecutively bound subunits, wherein the value of q for each successively bound subunit is 0, followed by 1, followed by 0, followed by 1, followed by 0, etc.

In one or more embodiments chiral centers present in the backbone of the polymer molecules retain the conformation present in the corresponding Dextran atoms from which the polymer molecules were prepared.

That is, the polymer molecules of the mixture comprise a series of consecutively bound subunits, wherein for each subunit, an adjacent subunit that is bound to the Oxygen atom in the backbone of the subunit is bound to a Carbon atom in the backbone of the adjacent subunit and wherein, an adjacent subunit that is bound to a Carbon atom in the backbone of the subunit is bound to the Oxygen atom in the backbone of the adjacent subunit.

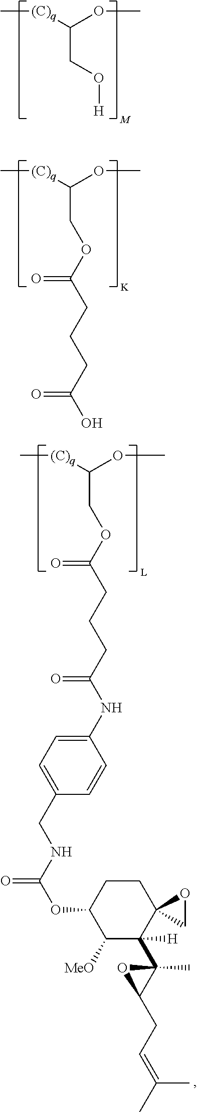

### Composition C

Composition C is a mixture of polymer molecules, wherein a polymer molecule in the mixture comprises a novel polymeric prodrug of fumagillin derivative, Compound B conjugated to PHF via ester and amide bonds and by a glutaric acid linker. Composition C can be synthesized in a multistep process wherein the PHF is derived from Dextran, a glutaric acid linker is conjugated to the PHF and the fumagillin derivative is conjugated to the glutaric acid linker.

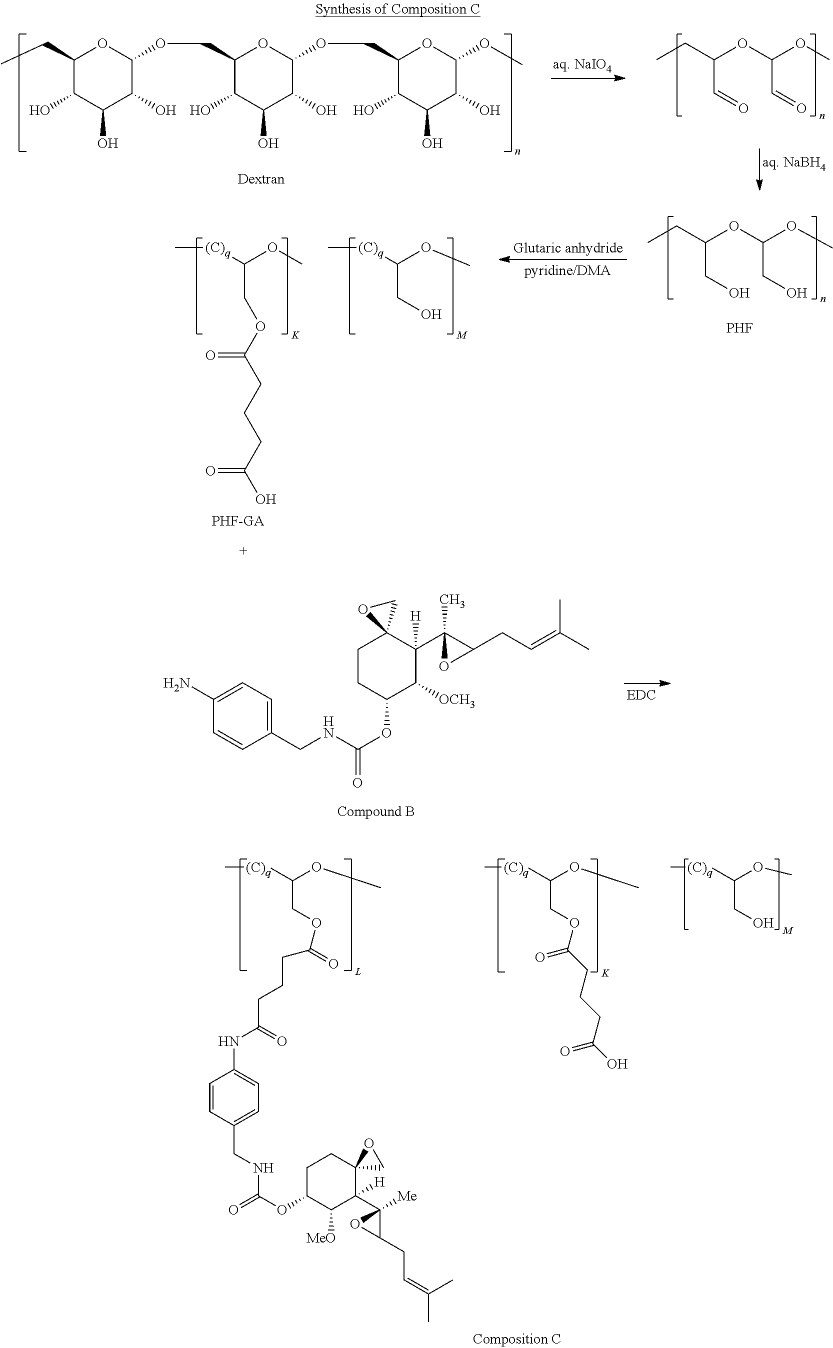

Compound B is conjugated to a glutaric acid linker on the PHF backbone. Additionally, glutaric acid conjugated residues that are not conjugated to Compound B impact physical properties of the product.

Dextran has multiple chiral centers including two from each dextran monomer which are retained in the backbone of PHF. Accordingly, in one or more embodiments of the invention, chiral centers present in the backbone of the polymer molecules retain the conformation present in the corresponding Dextran atoms from which the polymer molecules were prepared. In such embodiments, the chirality of the Dextran C5 and the a-configuration of Dextran C1 will be retained.

Dextran also has a directionality that is retained by the PHF backbone and for each of the subunits of PHF-GA and Composition C as shown. Accordingly, in one or more embodiments of the invention, polymer molecules of the mixture comprise a series of consecutively bound subunits, wherein for each subunit, an adjacent subunit that is bound to the Oxygen atom in the backbone of the subunit is bound to a Carbon atom in the backbone of the adjacent subunit and wherein, an adjacent subunit that is bound to a Carbon atom in the backbone of the subunit is bound to the Oxygen atom in the backbone of the adjacent subunit.

Monomers in Dextran contain a Carbon atom at one end corresponding to C6 and an Oxygen atom at the other end bound to C1. Accordingly, in one or more embodiments of the invention, PHF molecules will have a monomer where q=1 at one termini (containing backbone Carbon atoms corresponding to C5 and C6) and will have a monomer where q=0 at the other termini (containing a backbone Carbon atom corresponding to C1).

In one or more embodiments, the amount of glutaric acid in the PHF-GA of the invention is about 7% to about 16% glutaric acid by weight, about 8% to about 14% glutaric acid by weight, about 9% to about 13% glutaric acid by weight, or about 10.1% glutaric acid by weight, or about 12.2% glutaric acid by weight.

In one or more embodiments the Mol % of subunit K in the PHF-GA is about 3% to about 9.5%, about 4% to about 8%, or about 5% to about 6.5%, or about 5.6%, or about 6.9%.

The pH or the reaction in which Compound D is conjugated to the PHF-GA is a critical parameter for obtaining a product with desired physical, properties. Thus, in one or more embodiments acceptable for this invention, Compound B is reacted with PHF-GA at a pH of about 40 to about 6.0, about 4.2 to about 5.8, or about 4.2 to about 5.5, or about 5.5.

The primary release product, Compound D is slowly released from Composition C polymer backbone in vivo under physiological pH and/or by enzymatic hydrolysis of the ester bond between PHF and glutaric acid. Compound D is also the biologically active component of Composition C. By conjugating Compound D to PHF, both its in vivo anti-angiogenic and antitumor activities are enhanced. In addition, the conjugate Composition C showed superior pharmacokinetics due to slow release of Compound D from the polymer backbone of Composition C.

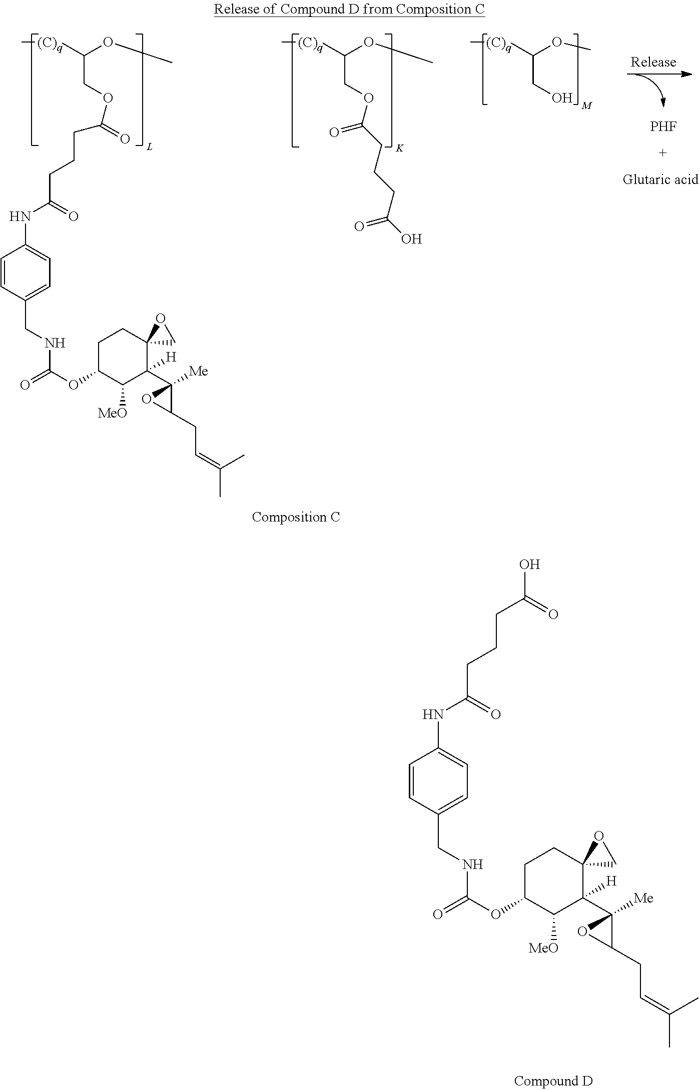

The amount of Compound D which is in Composition C plays a critical role in the utility of Composition C. If the amount is below the specified range it requires a great excess of Composition C to deliver sufficient Compound D (the primary and biological active release product) to have therapeutic effect. For example, a preparation of Composition C with 1% Compound D by weight, would require administration of 100 grams of Composition C in order to administer 1 gram of Compound D. To administer the same 1 g of Compound D from a preparation of Composition C with 10% Compound D by weight, only 10 grams of Composition C would be required. Therefore, higher levels of Compound D loading can deliver a greater amount of Compound D and could have been expected to be advantageous in a pharmaceutical formulation. However, it has been surprisingly discovered that if the loading is above the specified range, critical physical properties are negatively impacted such as aqueous solubility, viscosity, particle size, aggregation and molecular weight.

In one or more embodiments Compound D is about 9% to about 14% of Composition C by weight, about 10% to about 14% by weight, about 10.5% to about 14% by weight, about 1.1% to about 0.14% by weight, about 11.25% to about 13%, or about 11.5% to about 12.5%, or about 11.8%, or about 11.9%.

In one or more embodiments the Mol % of subunit L subunits in Composition C is about 1.2% to about 2.2%, about 1.4% to about 2.2%, about 1.6% to about 2.2%, about 1.4% to about 2.1%, about 1.5% to about 2.0%, about 1.64 to about 2.0%, or about 1.7% to about 1.9%, or about 1.75%, or about 1.80%.

The backbone of PHF contains acetals which tend to hydrolyze at low pH. In contrast, Fumagillol is attached to PHF via ester and amide linkages which tend to hydrolyze at high pH. Composition C therefore has components that are individually destabilized at both low pH and high pH. In addition, Composition C tends to form high molecular weight species if left unformulated in either aqueous solution or as a lyophilized powder. Therefore, it is formulated immediately upon completion of the synthesis. Formulation components can include buffering components and stabilizing agents.

Composition C aqueous solution is buffered to the desired pH using conventional buffers. Non-limiting examples of buffers suitable for use with the solutions include one or more of sodium citrate, ascorbate, succinate, lactate, citric acid, boric acid, borax, hydrochloric acid, disodium hydrogen phosphate, acetic acid, formic acid, glycine, bicarbonate, tartaric acid, Tris-glycine, Tris-NaCl, Tris-ethylenediamine tetraacetic acid (“EDTA”), Tris-borate-EDTA, Tris-acteate-EDTA (“TAB”) buffer and Tris-buffered saline, 4-(2-hydroxyethyl)-1-piperazineethanesulfonic acid (“HEPES”), 3-(N-morpholino)propanesulfonic acid (“MOPS”), piperazine-1,4-bis(2-ethanesulfonic acid) (“PIPES”), 2-(N-morpholino)ethanesulfonic acid (“MES”), and phosphate buffered saline (“PBS”).

In one embodiment, Composition C aqueous solution is buffered with a pH 5.5 buffer solution of sodium citrate and citric acid. In one embodiment, Composition C formulation contains approximately 8.4% of sodium citrate and 1.2% of citric acid by weight.

Non-limiting examples of stabilizing agents suitable for use with the formulations include mannitol, sorbitol, polyvinyl pyrrolidone, sucrose, lactose, glucose, xylitol, maltose, fructose, raffinose, galactose, trehalose, hydroxypropyl-β-cyclodextrin, and lactitol.

In some embodiments, Composition C aqueous solution may contain additional components.

In one or more embodiments, Composition C aqueous solution may contain soluble or insoluble additives typically found in pharmaceutical formulations. Non-limiting examples of additives useful with the aqueous solutions include pharmaceutically acceptable excipients such as surfactants, anti-humiditants, anti-oxidants, viscosifiers, salts, and preservatives.

In one or more embodiments, the aqueous solution may contain a surfactant or a mixture of surfactants including but not limited to Polysorbate 80, Polysorbate 20, Sorbitan esters, PEG stearates, Poloxamer 407, Solutol HS 15, Poloxamer 188, Tween 80, sodium lauryl sulphate, ether sulphates, sulphated oils, cetrimide BP, benzalkonium chloride, lecithin, cetromacrogel 1000 BPC, and alkali metal soaps of the formula RCOOX where R=C₁₀-C₂₀ alkyl group, and X=sodium, potassium, or ammonium.

In one or more embodiments, the aqueous solution may contain a preservative or a mixture of: preservatives including but not limited to benzyl alcohol, sodium benzoate acid, sodium nitrate, sulphur dioxide, sodium sorbate and potassium sorbate.

In one or more embodiments, Composition C aqueous solution is sterile. Filtration is a non-limiting example of sterilization methods useful with the aqueous solution. In some embodiments, the aqueous solution is sterilized by filtration through a 0.1 micron and/or 0.2 micron filter.

Pharmaceutical compositions are often lyophilized for transport and are reconstituted immediately before use. However, Composition C has been observed to form high molecular weight species, in some cases irreversibly. The invention herein provides Composition C with the correct ratios of subunits which eliminate, or at least minimize the formation of the high molecular weight species. Additionally, in some embodiments, the lyophilized formulation contains a stabilizing agent that allows the lyophilized formulation to be reconstituted. Non-limiting examples of stabilizing agents suitable for use with the lyophilized formulations include mannitol, sorbitol, polyvinyl pyrrolidone, sucrose, lactose, glucose, xylitol, maltose, fructose, raffinose, galactose, trehalose, hydroxypropyl-β-cyclodextrin, and lactitol.

In one embodiment, the lyophilized formulation contains 35-50% mannitol by weight; in another embodiment the lyophilized formulation contains about 42% mannitol by weight.

In some embodiments, the lyophilized formulation contains less than about 4% water by weight. Therefore, in some embodiments the lyophilized formulation has a pH of about pH 5.0 to about 6.0. In other embodiments the lyophilized formulation has a pH of about pH 5.5. The pH of the lyophilized formulation is controlled by the use of buffers. Non-limiting examples of buffers suitable for use with the formulations include one or more of sodium citrate, ascorbate, succinate, lactate, citric acid, boric acid, borax, hydrochloric acid, disodium hydrogen phosphate, acetic acid, formic acid, glycine, bicarbonate, tartaric acid, Tris-glycine, Tris-NaCl, EDTA, TAP buffer and Tris-buffered saline, HEPES, MOPS, PIPES, MES, and PBS. In some embodiments, the lyophilized formulation contains about 8.4% sodium citrate and 1.2% citric acid by weight. The buffer may be selected to provide pH stability in both Composition C aqueous solution prior to lyophilization and the lyophilized dry formulation.

The lyophilized formulation is suitable for intravenous administration after reconstitution. Suitable agents for reconstitution include but are not limited to sterile water for injection, USP and 0.9% Sodium Chloride Injection, USP.

As used herein, “about” with regard to a stated number encompasses a range of +2 percent to −2 percent of the stated value. By way of example, about 100 mg/kg therefore includes the range 98-102 mg/kg and therefore also includes 98, 99, 100, 101 and 102 mg/kg. Accordingly, about 100 mg/g includes, in an embodiment, 100 mg/kg.

It is understood that where a parameter range is provided, all integers within that range, tenths thereof, and hundredths thereof, are also provided by the invention. For example, “0.2-5 mg/kg” is a disclosure of 0.2 mg/kg, 0.21 mg/kg, 0.22 mg/kg, 0.23 mg/kg etc. up to 0.3 mg/kg, 0.31 mg/kg, 0.32 mg/kg, 0.33 mg/kg etc. up to 0.4 mg/kg, 0.5 mg/kg, 0.6 mg/kg etc. up to 5.0 mg/kg.

All combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Methods

### Amount of Unbound Glutaric Acid

The amount of unbound glutaric acid in Composition C was determined using reverse phase HPLC with UV detection. The UV detector was set; at 203 nm. The level of unbound glutaric acid in the sample was quantified by peak area comparison to the glutaric acid standard.

### Determination of Glutaric Acid Loading in PHF-GA

The amount of glutaric acid (GA) loaded (covalently bound) to PHF-GA was verified by quantitative hydrolysis of glutaric acid from the polymer backbone followed by reverse phase HPLC(RP-HPLC). UV absorbance at 203 nm is measured and a GA standard was used for calculations. The amount of GA in the hydrolyzed sample is corrected for the amount of unbound glutaric acid present in PHF-GA according to the following function:

GA loading=GA weight measured after hydrolysis−GA weight measured before hydrolysis.

Weight % GA in PHF-GA is calculated according to the following function:

$$Weight \% GA = 100 \times \frac{Concentration bound GA}{Concentration PHF \text{-} GA} ;$$

where a desired Concentration of PHF-GA is obtained by dissolving a known amount of lyophilized PHF-GA and adjusting the volume to achieve the desired concentration; and

Mol % GA is determined by the function:

$$Mol \% GA = \frac{100 * ( weight \% GA / Mw GA )}{\begin{matrix}( 2 * ( ( 100 - weight \% GA ) + \\ ( weight \% GA / Mw GA ) * Mw {H}_{2} O ) / Mw PHF )\end{matrix}} ;$$

where Mw PHF is the molecular weight of a monomer of PHF according to Formula I 134.13 g/mol, Mw GA 132.11 g/mol, and Mw H₂O 18.02 g/mol.

Mol % GA is equivalent to mol % subunit K in PHF-GA.

The amount of glutaric acid covalently bound to PHF-GA is measured following Phase 3 of Example 1, below. Subunit K in PHF-GA represents sites available for conjugation of compound B in Example 5 below and accordingly affects both the amount of conjugated compound B and the amount of GA not conjugated to Compound B in Composition C.

### Determination of Compound D-Related Impurities in Composition C by (RP-HPLC)

The free Compound D and other impurities in Composition C were measured by RP-HPLC by injecting samples of Composition C and a Compound D standard. Analytes were separated by their retention within the column and measured for total area under the curve by UV absorbance at 247 nm. The level of free Compound D and other impurities in the sample were quantified by comparison of individual peak areas to peak areas for known Compound D standards. Detection limits were <0.05%.

### UV Assay for Measurement of Compound D Equivalents

Compound D loading in Composition C was determined by measuring the optical density at 247 nm with a background correction set at 500 nm. Total amount of Compound D was determined by calculation using the extinction coefficient and dilution factor and the amount of bound Compound D was determined by correction for any free Compound D impurities observed in Composition C using the method described above. Compound D weight % was then calculated relative to the total concentration of Composition C conjugate in solution according to the following formula:

Weight % Compound D=(OD₂₄₇₋₅₀₀×Mw×DF)/(ε₂₄₇×C_{Composition C})×100
where OD₂₄₇₋₅₀₀=OD₂₄₇−OD₅₀₀, is absorption at 247 nm corrected for background at 500 nm;
ε₂₄₇ is Compound D extinction coefficient for λ=247 nm, =15000 [L/mol·cm];
Mw is the molecular weight of Compound D=544.64 g/mol;
C_{Composition C} is Composition C concentration, mg/mL; and
DF=Sample Dilution Factor.

Concentration of Composition C is determined by a Size-Exclusion Chromatography method using a refractive index detector and based on comparison of Composition C conjugate peak area in sample with Composition C peak area in a Composition C standard.

Weights Compound B is determined by the function:

$$Weight \% Compound B = Weight \% Compound D \times \frac{Mw Compound B}{Mw Compound D} ;$$

and
where Mw Compound B=430.54 g/mol and Mw Compound D=544.64 g/mol.

Mol % Compound D is determined by the function:

$$Mol \% Compound D = \frac{100 * ( weight \% Compound B / Mw Compound B )}{\begin{matrix}( 2 * ( ( 100 - weight \% Compound B ) + ( weight \% \\ Compound B / Mw Compound B ) * Mw {H}_{2} O ) / Mw PHF \text{-} GA )\end{matrix}} ,$$

where Mw PHF-GA is an average molecular weight of a subunit of PHF-GA as calculated by the function:

$$Mw PHF \text{-} GA = \frac{\begin{matrix}( ( 2 * Mol \% GA * ( Mw PHF + Mw glutaric acid - Mw {H}_{2} O ) ) + \\ ( ( 100 - 2 * Mol \% GA ) * Mw PHF )\end{matrix}}{100} ;$$

and where Mw PHF is the molecular weight of a monomer of PHF according to Formula I.

In one or more embodiments subunit K subunits of Composition C represent subunit K subunits of PHF-GA which are not conjugated to Compound B. The Mol % of subunit K in Composition C depends directly on the Mol % of subunit K in the PHF-GA and the Mol % of subunit L in Composition C according to the following formula:

Mol % subunit K in Composition C=Mol % subunit K in PHF-GA−Mol % subunit L in Composition C.

Determination of Mol % Ratios in Composition C

| Subunit | Description | Calculation |
|---|---|---|
| L | Compound D | Calculated from measured Compound |
| | loading | D in composition C |
| K | Conjugated | Subunit K in PHF-GA − Subunit L in |
| | Glutaric Acid | Composition C, where subunit K in |
| | | PHF-GA is calculated from measured |
| | | GA in PHF-GA |
| M | Unconjugated | Total subunits (100) − Subunit K in |
| | (contains free | PHF-GA, where subunit K in PHF-GA is |
| | hydroxyl group) | calculated from measured GA in PHF-GA |

### Molecular Weight Distributions (Mw, D₉₀, D₅₀, D₁₀)

The molecular weight distributions of Composition C conjugate, PHF and PHF-GA were measured by high performance size exclusion chromatography (HPSEC) with RI detection. Separation was carried out on a GE Healthcare Superose 6 column using 50 mM pH=7.4 phosphate 0.9% NaCl as an fluent. Dextran standards (American Polymer Standards Corporation) were used to establish a calibration curve of known molecular weight vs. retention time. Molecular weight distributions (weight average molecular weight (“Mw”), D₉₀, D₅₀, D₁₀) were calculated based on the polysaccharide standard curve.

### Particle Size

Particle size of Composition C conjugates was measured with HPSEC with Wyatt miniDawn Trees light scattering detector and Optilab RI detector.

### Viscosity

Viscosity Composition C conjugates was measured on the HAAKE RotoVisco 1 viscometer with D=60 mm, 1°, titanium cone as sensor. Viscosity is the mean value of a relatively flat portion of the curve, in which viscosity is independent of the shear rate.

### Osmolality

The osmolality of aqueous Composition C was measured by a vapor pressure osmometer (Vapor).

### Example 1

### Production of PHF-GA

### Phase 1

### Oxidation of Dextran

Dextran is subjected to exhaustive oxidation in aqueous sodium periodate (NaIO₄) to yield a polymeric poly-aldehyde in which the carbon at position three of each glucose residue has been excised. The oxidized dextran is desalted first by vacuum filtration to remove precipitated inorganic salts and then by diafiltration using a filter having a nominal Mw cut off (MWCO) of 10 kDa.

### Phase 2

### Synthesis of PHF

The purified poly-aldehyde is then exhaustively reduced using aqueous sodium borohydride (NaBH₄) to yield poly[hydroxymethylethylene hydroxymethylformal], an alternating co-polymer of glycol aldehyde and glycerol, abbreviated ‘PHF’. The PHF is purified by diafiltration using a filter having a nominal MWCO of 10 kDa. The purified PHF is filtered through a 0.2 micron filter, lyophilized to a solid, and stored at 2-8° C.

### Phase 3

### Synthesis of PHF-GA

The free hydroxyls of the PHF are glutarated using glutaric anhydride in a mixture of pyridine and dimethylacetamide (DMA) to yield PHF-GA. The PHF-GA is then purified by diafiltration using a filter having a nominal MWCO of 10 kDa. GA loading is controlled by the amounts of PHF and glutaric anhydride used in the reaction and is verified as described above.

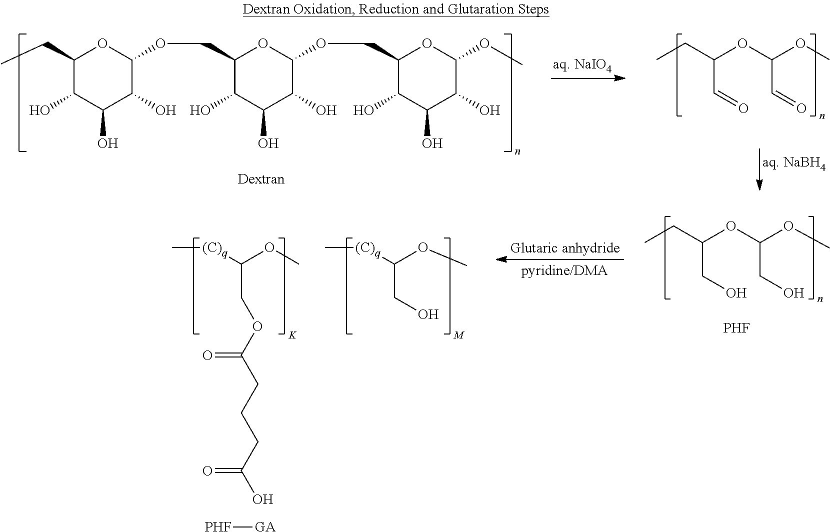

### Example 2

### Hydrolysis of Fumagillin to Fumagillol

Fumagillol is prepared in a single step from fumagillin via hydrolysis. Fumagillin dicyciohexylammonium salt is hydrolyzed with 0.2N NaOH solution in presence of ether as a Diphasic mixture. The ether layer is separated, washed with 10% citric acid, and then evaporated in vacuo to afford Fumagillol as a red-brown oil.

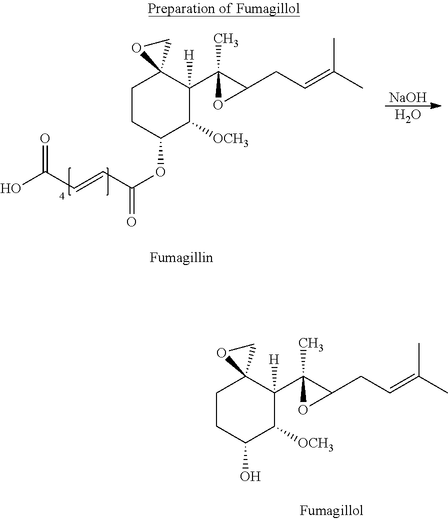

### Example 3

### Preparation of Compound A

Fumagillol is subsequently converted to its p-nitrophenylchloroformate derivative Compound A using triethylamine and dimethylaminopyridine in dichloromethane. Impurities are removed using column chromatography.

### Example 4

### Preparation of Compound B

Purified Compound A is reacted with p-aminobenzylamine in dichloromethane to afford Compound B. Compound B is then purified by column chromatography.

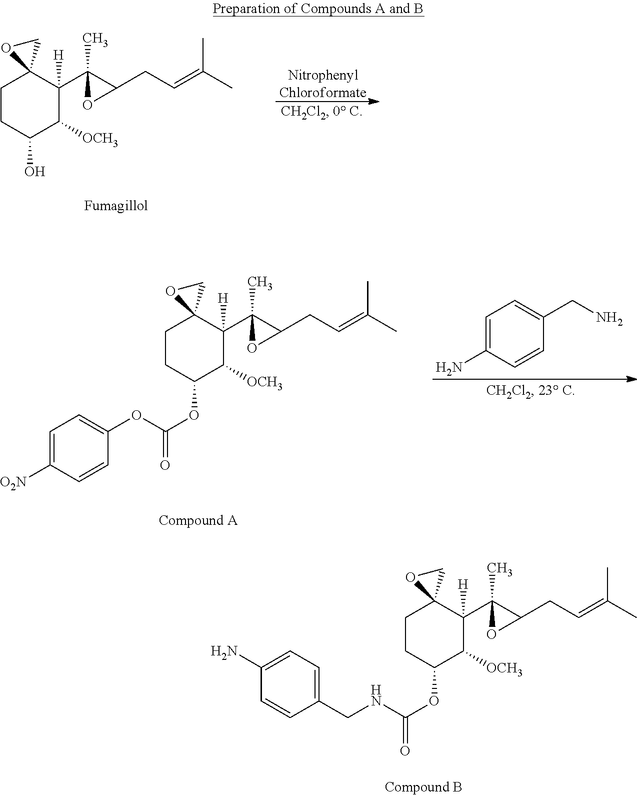

### Example 5

### Preparation of Composition C

A solution of Compound B in DMF is added to an aqueous solution of PHF-GA which contains about 10% DMA and the resulting mixture is cooled to <10° C. Ethyl dimethylaminopropyl carbodiimide (EDC) is added over a period of 10 to 15 minutes to activate the carboxylic acid group of PHF-GA. During addition and throughout the course of the reaction, the pH is maintained between about 4.0 and about 6.0 by adding either sodium bicarbonate or sulfuric acid mono sodium salt as appropriate. The mixture is stirred for 2.5 to 20 hours at room temperature. This yields an aqueous solution of Composition C. The aqueous solution of Composition C is filtered through a 0.2 μM membrane and then is purified by diafiltration using a filter having a nominal MWCO of 10 kDa. The purified Composition C is repeatedly subjected to diafiltration until a satisfactory concentration is achieved. The concentration is calculated by lyophilizing a measured amount of the aqueous solution and weighing the residue. The amount of Compound D which is conjugated is determined by an UV assay. The targeted amount of Compound D conjugated to the polymer is about 10.5% to about 17% by weight.

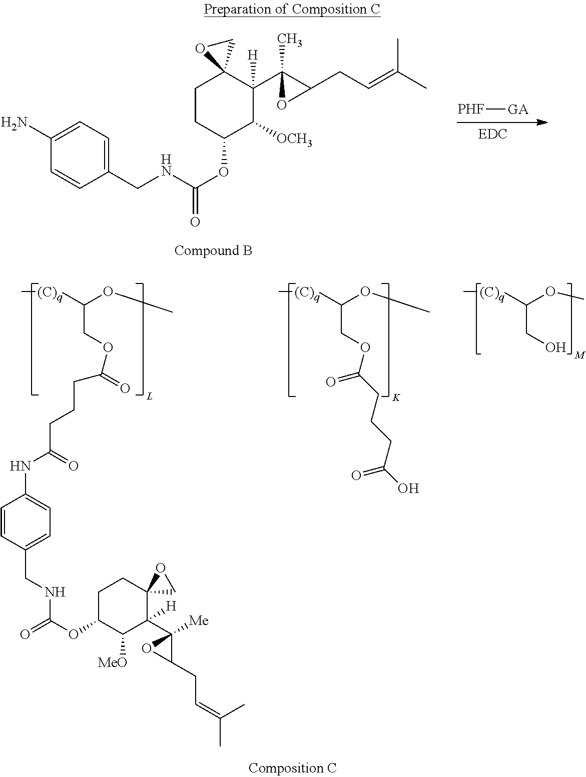

### Example 6

### Selection of Compound D Loading Levels on Various Preparations of Composition C

Compound B was conjugated to PHF-GA as described in Example 5. Multiple batches of PHF-GA were prepared as described in Example 1, Phase 3, with varying levels of GA conjugation and Composition C was prepared from each as described in Example 5. The amount of Compound B in the synthesis of Example 5 also was varied to yield conjugates with differing Compound D loading.

| | | Mol % | Weight % | Mol % | Mol % |
|---|---|---|---|---|---|
| Compo- | Weight % | subunit | Compound B | Subunit L | Subunit K |
| sition C | GA in | K in | in Compo- | in Compo- | in Compo- |
| Batch | PHF-GA | PHF-GA | sition C | sition C | sition C |
| A | 10.06 | 5.59 | 14.85 | 2.95 | 2.64 |
| B | 10.06 | 5.59 | 9.36 | 1.75 | 3.84 |
| C | 10.06 | 5.59 | 3.14 | 0.55 | 5.04 |
| D | 12.28 | 6.97 | 15.00 | 3.05 | 3.92 |
| E | 12.28 | 6.97 | 9.42 | 1.80 | 5.17 |
| F | 12.28 | 6.97 | 3.23 | 0.58 | 6.40 |
| G | 14.37 | 8.33 | 17.22 | 3.72 | 4.60 |

### Example 7

### Physical Properties of Composition C Batches

Physical properties of select Composition C batches were measured as described in the Methods section.

Solution Appearance of Different
Compound D Loading Conjugates

| Composition | Compound D Loading | Solution |
|---|---|---|
| C Batch | (Mol % Subunit L) | Appearance |
| D | 3.05 | Very slight |
| | | turbidity |
| E | 1.80 | Clear |
| F | 0.58 | Clear |

Preferred batches of Composition C did not exhibit turbidity in solution.

Viscosity of Different Compound B Loading Conjugates

| Composition | Compound D Loading | Viscosity at |
|---|---|---|
| C Batch | (Mol % Subunit L) | 50 mg/mL (cP) |
| A | 2.95 | 7.1 |
| B | 1.75 | 4.2 |
| C | 0.55 | 3.6 |

Particle Size of Different Compound B Loading Conjugates

| Composition | Compound D Loading | Particle Size |
|---|---|---|
| C Batch | (Mol % Subunit L) | (Radius, nm) |
| A | 2.95 | 10-20 (majority fraction) |
| D | 3.05 | 30-100 (small fraction) |
| | | <10 (small fraction) |
| B | 1.75 | 10-20 (small fraction) |
| E | 1.80 | 30-100 (a few particles) |
| | | <10 (majority fraction) |
| C | 0.55 | 10-20 (small fraction) |
| F | 0.58 | 30-100 (a few particles) |
| | | <10 (majority fraction) |

The particle size for the majority fraction of preferred batches of Composition C was <10 nm.

Molecular Weight Distribution of Different
Compound D Loading Conjugates

| | Compound D | Free Glutaric | |
|---|---|---|---|
| Composition | Loading (Mol % | Acid (Mol % | |
| C Batch | Subunit L) | Subunit K) | SEC Trace |
| A | 2.95 | 2.64 | 2 peaks (high |
| | | | molecular weight) |
| D | 3.05 | 3.92 | 2 peaks (high |
| | | | molecular weight) |
| B | 1.75 | 3.84 | Single peak |
| E | 1.80 | 5.17 | Single peak |
| C | 0.55 | 5.04 | Single peak |
| F | 0.58 | 6.40 | Single peak |

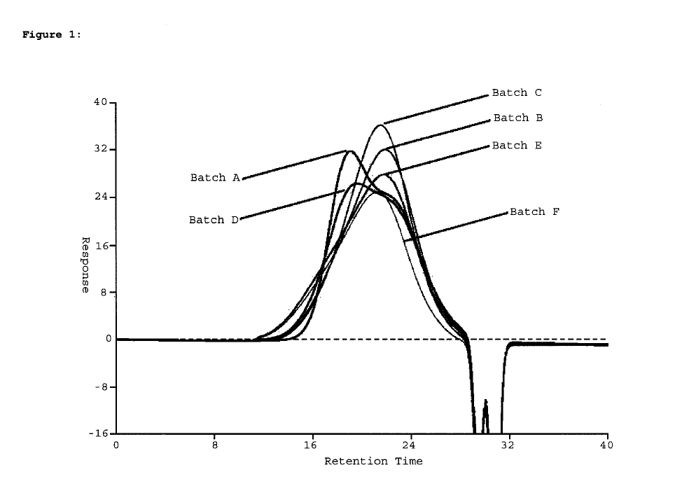

Composition C batches were analyzed by high performance size exclusion chromatography. Lower Compound D loading resulted in a single peak while 2 peaks were observed for higher Compound D loading samples. Results are shown in FIG. 1. Batches B, C, E and F displayed the desired physical characteristic of a single peak. Batches B and E have the additional desirable property of relatively high Compound D loading; approximately 3-fold higher than Batches C and F.

### Concentration of Different Compound D Loading Conjugates

Peak molecular weight values were measured for Composition C batches in solution at ˜3 mg/ml and at ˜60 mg/ml. Preferred batches of Composition C displayed apparent molecular weights that were concentration independent.

| Composition | Mp at ~3 | Mp at ~60 | |
|---|---|---|---|
| C Batch | mg/ml (kDa) | mg/ml (kDa) | % Change |
| A | 50091 | 101822 | +103 |
| B | 57480 | 55572 | −3.3 |
| C | 65360 | 61135 | −6.5 |
| D | 57819 | 91217 | +58 |
| E | 58840 | 57706 | −1.9 |
| F | 68130 | 64884 | −4.8 |
| G | 55209 | 110627 | +100 |

Preferred Compound D loading: 1.2-2.2 mol %.
Desired Compound D loading: 1.6-2.0 mol %.

### Example 8

### pH Dependent Stability of Composition C in Aqueous Solution

The backbone of PHF contains acetals which tend to hydrolyze at low pH. In contrast, Fumagillol is attached to PHF via ester and amide linkages which tend to hydrolyze at high pH. Composition C therefore has been found to become destabilized at both low pH and high pH.

Based on the observed apparent molecular weight (Mw) and molecular weight distribution (D₉₀, D₅₀, D₁₀) results, the polymer backbone was most stable at pH 5.5±0.2 and 6.5.2. However, at pH 6.5±0.2, a significant amount of Compound D was released from the polymer backbone at day 6 as compared to day 0 (0.26% vs. 0.01%). Therefore, pH about 5.5 was selected as an appropriate range for formulation.

Stability of Composition C Solution at
Different pH at Ambient Temperature

| Tar- | Time | | | | |
|---|---|---|---|---|---|
| geted | Point | Measured | Appear- | | |
| pH | (Day) | pH | ance | SEC (kDa) | Impurity (%) |
| 4.5 ± 0.2 | 0 | 4.44 | Clear | Mw: 159 | None |
| | | | solution | D₉₀: 400 | detected |
| | | | | D₅₀: 98 | |
| | | | | D₁₀: 34 | |
| | 3 | 4.60 | Clear | Mw: 145 | N/A |
| | | | solution | D₉₀: 354 | |
| | | | | D₅₀: 91 | |
| | | | | D₁₀: 33 | |
| | 6 | 4.54 | Clear | Mw: 128 | Compound |
| | | | solution | D₉₀: 302 | D: 0.04 |
| | | | | D₅₀: 81 | RRT 0.87: |
| | | | | D₁₀: 29 | 0.01 |
| 5.5 ± 0.2 | 0 | 5.48 | Clear | Mw: 160 | Compound |
| | | | solution | D₉₀: 400 | D: <0.01 |
| | | | | D₅₀: 98 | |
| | | | | D₁₀: 34 | |
| | 3 | 5.77 | Clear | Mw: 159 | N/A |
| | | | solution | D₉₀: 391 | |
| | | | | D₅₀: 97 | |
| | | | | D₁₀: 34 | |
| | 6 | 5.85 | Clear | Mw: 156 | Compound |
| | | | solution | D₉₀: 390 | D: 0.03 |
| | | | | D₅₀: 95 | |
| | | | | D₁₀: 33 | |
| 6.5 ± 0.2 | 0 | 6.67 | Clear | Mw: 159 | Compound |
| | | | solution | D₉₀: 400 | D: 0.01 |
| | | | | D₅₀: 96 | |
| | | | | D₁₀: 34 | |
| | 3 | 6.95 | Clear | Mw: 161 | N/A |
| | | | solution | D₉₀: 404 | |
| | | | | D₅₀: 99 | |
| | | | | D₁₀: 35 | |
| | 6 | 7.29 | Clear | Mw: 158 | Compound |
| | | | solution | D₉₀: 390 | D: 0.26 |
| | | | | D₉₀: 97 | |
| | | | | D₁₀: 33 | |

Preferred pH range: 5-6.
Desired pH: 5.5

### Example 9

### Formulation of Composition C

In the following example a citrate butter was selected to improve Composition C stability and a mannitol stabilizing agent was selected to overcome the known problem of formation of high molecular weight species of Composition C.

An aqueous Composition C conjugate was formulated with a sodium citrate dihydrate/citric acid monohydrate buffer solution, mannitol, and water for injection to yield the stabilized aqueous solution described in the following Table. Mannitol is used as a stabilizing agent to prevent formation of high molecular weight species of Composition C and to facilitate re-constitution of the lyophilized Composition C.

Composition of Formulated Aqueous Conjugate with Mannitol

| Component | Approximate Dry Weight % |
|---|---|
| Compound D equivalents | 5.8% |
| Composition C conjugate | 48.7% |
| Mannitol | 41.7% |
| Sodium citrate | 8.4% |
| Citric acid | 1.2% |

Preferred amount of mannitol is 35-50% by weight.

Desired amount of mannitol is approximately 42% by weight.

The formulated Composition C solution was then 0.1 micron or 0.2 micron filtered and packaged in sterile polycarbonate carboy and stored at 2° C. to 8° C. or −20° C.

Stability of aqueous formulations of Composition C was measured at 2-8° C. and −20° C.

Stability of Composition C Aqueous Formulation at 2-8° C.

| Test | T = 0 | T = 1 month | T = 2 month | T = 3 month |
|---|---|---|---|---|
| Appearance | Clear | Clear | Clear | Clear |
| (Visual) | Solution | Solution | Solution | Solution |
| pH | 5.5 | 5.4 | 5.5 | 5.5 |
| Molecular Weight | Mw: 97 | Mw: 108 | Mw: 112 | Mw: 116 |
| Analysis/ | D90: 203 | D90: 236 | D90: 248 | D90: 265 |
| Distribution | D50: 70 | D50: 74 | D50: 75 | D50: 76 |
| (kDa) | D10: 30 | D10: 30 | D10: 29 | D10: 28 |
| Impurities | RRT 0.31: | RRT 0.31: | RRT 0.31: | RRT 0.31: |
| (≧0.05%) | 0.08% | 0.08% | 0.08% | 0.07% |

The apparent molecular weight of Composition C was observed to increase over time when stored at 2-8° C. A lyophilized product was selected as a solution to this problem.

### Example 10

### Lyophilization of Composition C Formulations

In the following Example the aqueous solution from Example 8 was selected for lyophilization. Each vial (30 mL) was filled with about 15 mL of the aqueous solution from Example 8 above, and then lyophilized by the following lyophilization cycle to generate lyophilized cakes. After the lyophilization cycle was finished, the vials were stopped to 95% atmosphere with (pure) nitrogen.

Lyophilized formulations containing 54% water by weight were stored at 2-8° C. for up to 10 months and physical properties were measured to assess stability.

Stability of Composition C Lyophilized Formulation at 2-8° C.

| | | T = 1 | T = 3 | T = 4 | T = 6 | T = 10 |
|---|---|---|---|---|---|---|
| Test | T = 0 | month | months | months | months | months |
| Appearance | White | White | White | White | White | White |
| | solid | solid | solid | solid | solid | solid |
| pH | 5.5 | 5.4 | 5.5 | 5.4 | 5.4 | Not |
| | | | | | | tested |
| Osmolality | 286 | 280 | Not | 290 | 290 | Not |
| (mOsm/kg) | | | tested | | | tested |
| Molecular | 100 | 102 | 98 | 98 | 101 | 111 |
| Weight | | | | | | |
| Mw (kDa) | | | | | | |
| Molecular | D₁₀ = 30 | D₁₀ = 30 | D₁₀ = 28 | D₁₀ = 29 | D₁₀ = 27 | D₁₀ = 33 |
| Weight | D₅₀ = 71 | D₅₀ = 72 | D₅₀ = 70 | D₅₀ = 70 | D₅₀ = 69 | D₅₀ = 74 |
| Distribution | D₉₀ = 209 | D₉₀ = 215 | D₉₀ = 209 | D₉₀ = 206 | D₉₀ = 214 | D₉₀ = 225 |
| (kDa) | | | | | | |
| Composition C | 675 | 684 | Not | 719 | 698 | Not |
| (mg/vial) | | | tested | | | tested |
| Compound D | 75 | 75 | Not | 77 | 77 | Not |
| Equivalents | | | tested | | | tested |
| (mg/vial) | | | | | | |
| Impurities | None | None | None | None | None | None |
| (≧0.05%) | ≧0.05% | ≧0.05% | ≧0.05% | ≧0.05% | ≧0.05% | ≧0.05% |

Lyophilized Composition C was shown to be stable for long term storage. Accordingly, lyophilized Composition C containing ≦4% water by weight was selected as a composition containing Composition C.

### Example 11

### Reconstitution of Lyophilized Composition C Formulations

The following example demonstrates that the selected formulation of Composition C successfully overcomes the irreversible agglomerization observed for lyophilized Composition C. Sterile water for injection, USP and 0.9% Sodium Chloride Injection, USP were selected as reconstitution agents for preparation of an injectable formulation suitable for intravenous administration.

### Reconstitution in Sterile Water

A lyophilized formulation containing about 675 mg Composition C in a 20 mL vial was reconstituted with about 15 ml of sterile water for injection, USP, resulting in an isotonic solution with an osmolality of 285 mOsmol/kg.

### Reconstitution in Sodium Chloride Solution

A lyophilized formulation containing about 222 mg Composition C in a 30 ml, vial was reconstituted with about 15 mL 0.9% Sodium Chloride Injection, USP, resulting in an isotonic solution with an osmolality of 360 mOsmol/kg.

What is claimed is:

1. A mixture comprising polymer molecules or salts thereof, wherein a polymer molecule in the mixture comprises covalently bound subunits L, K, and M represented as: 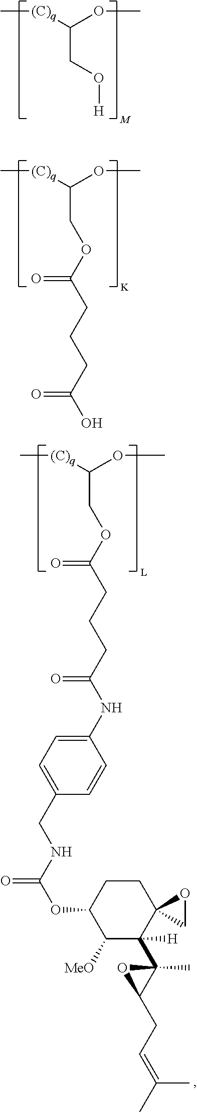
wherein q=0 or 1,
wherein every subunit which is bound to a subunit where q is 1 is a subunit where q is 0 and every subunit which is bound to a subunit where q is 0 is a subunit where q is 1 such that subunits where q is 0 and subunits where q is 1 alternate in the polymer molecule,
wherein the average molecular weight of the polymer molecules in the mixture is about; 50 kDa to about 200 kDa,
wherein the mole percentage of subunit M, relative to the total amount of subunits in the mixture, is about 91.5 to about 96 mol %,
wherein the mole percentage of subunit K, relative to the total amount of subunits in the mixture, is about 2.8 to about 7.3 mol %, and
wherein the mole percentage of subunit L, relative to the total amount of subunits in the mixture, is about 1.2 to about 2.2 mol %.

2. The mixture of claim 1, wherein the mole percentage of subunit M, relative to the total amount of subunits in the mixture, is about 93.5 to about 95 mol %.

3. The mixture of claim 1, wherein the mole percentage of subunit K, relative to the total amount of subunits in the mixture, is about 2.8 to about 6.0 mol %

4. The mixture of claim 3, wherein the mole percentage of subunit K, relative to the total amount of subunits in the mixture, is about 2.8 to about 4.9 mol %.

5. The mixture of claim 1, wherein the mole percentage of subunit L, relative to the total amount of subunits in the mixture, is about 1.6 to about 2.2 mol %.

6. A mixture comprising polymer molecules or salts thereof, wherein a polymer molecule in the mixture comprises a poly(1-hydroxymethylethylene hydroxymethyl-formal) backbone having covalently bound thereto through a carboxyl group, glutaric acid and Compound D of the formula: 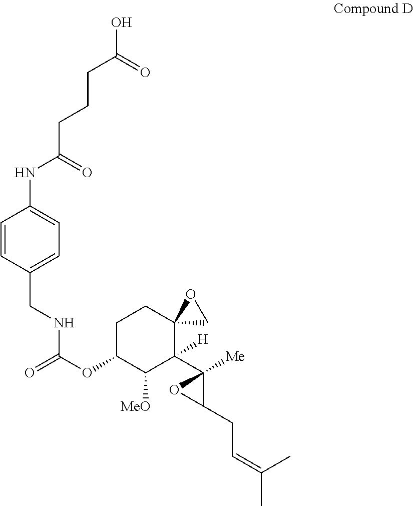
wherein the average molecular weight of the polymer molecules in the mixture is about 50 kDa to about 200 kDa,
wherein the mole percentage of glutaric acid covalently bound to the mixture of polymer molecules, relative to the total amount of subunits in the mixture, is about 2.8 to about 7.3 mol %, and
wherein the mole percentage of Compound D covalently bound to the mixture of polymer molecules, relative to the total amount of subunits in the mixture, is about 1.2 to about 2.2 mol %.

7. The mixture of claim 6, wherein the mole percentage of glutaric acid covalently bound to the mixture of polymer molecules, relative to the total amount of subunits in the mixture, is about 2.8 to about 6.0 mol %.

8. The mixture of claim 7, wherein the mole percentage of glutaric acid covalently bound to the mixture of polymer molecules, relative to the total amount of subunits in the mixture, is about 2.8 to about 4.9 mol %.

9. The mixture of claim 6, wherein the mole percentage of Compound D covalently bound, to the mixture of polymer molecules, relative to the total amount, of subunits in the mixture, is about 1.6 to about 2.2 mol %.

10. The mixture of claim 1, wherein the average molecular weight of the polymer molecules in the mixture is about 70 kDa.

11. The mixture of claim 1, wherein the peak molecular weight is less than 100 kDa.

12. The mixture of claim 1 wherein the molecular weight distribution of the mixture of polymer molecules has a single peak.

13. The mixture of 1 wherein the peak molecular weight is less than 70 kDa.

14. The mixture of claim 13, wherein the peak molecular weight is about 40 kDa to about 60 kDa.

15. The mixture of 1, wherein D₁₀ of the molecular weight distribution of the mixture of polymer molecules is less than or equal to 50 kDa.

16. The mixture of 1, wherein D₅₀ of the molecular weight distribution of the mixture of polymer molecules is less than or equal to 200 kDa.

17. The mixture of claim 1, wherein D₉₀ of the molecular weight distribution of the mixture of polymer molecules is less than or equal to 300 kDa.

18. The mixture of claim 1, further comprising one or more impurities, wherein the one or more impurities are present in an amount of less than 5% by weight.

19. The mixture of claim 18, wherein the impurities are present in an amount of about 1% to about 5% by weight.

20. The mixture of claim 1, wherein the salt is a pharmaceutically acceptable salt.

21. A pharmaceutical formulation comprising the mixture of claim 1.

22-43. (canceled)

44. A process for producing a mixture comprising polymer molecules, wherein a polymer molecule in the mixture comprises covalently bound subunits L, K, and M represented as: 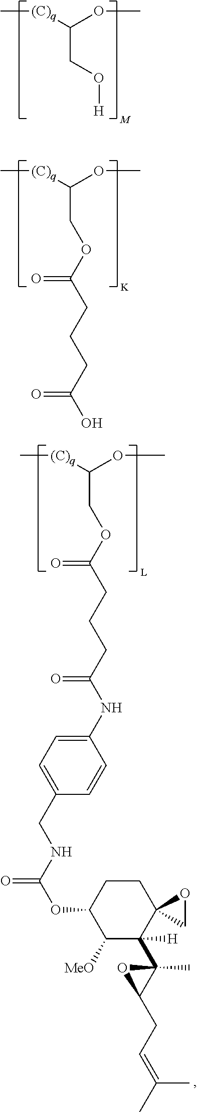
wherein q=0 or 1,
wherein every subunit which is bound to a subunit where q is 1 is a subunit where q is 0 and every subunit which is bound to a subunit where q is 0 is a subunit where q is 1 such that subunits where q is 0 and subunits where q is 1 alternate in the polymer molecule,
wherein the average molecular weight of the polymer molecules in the mixture is about 50 kDa to about 200 kDa,
wherein the mole percentage of subunit M, relative to the total amount of subunits in the mixture, is about 91.5 to about 96 mol %,
wherein the mole percentage of subunit K, relative to the total amount of subunits in the mixture, is about 2.8 to about 7.3 mol %, and
wherein the mole percentage of subunit. L, relative to the total amount of subunits in the mixture, is about 1.2 to about 2.2 mol %, the process comprising,
a) obtaining a mixture of PHF-GA molecules having at least 3 mole percentage of subunit K, relative to the total amount of subunits in the mixture of PHF-GA molecules,
b) reacting Compound B with the mixture of PHF-GA molecules,
thereby producing the mixture comprising the polymer molecules.

45-49. (canceled)

50. A method of treating cancer or an angiogenic disease, comprising administering to a subject in need thereof a mixture of claim 1 in an amount effective to treat the cancer or the angiogenic disease.

51-56. (canceled)

57. A method of delivering Compound D of the formula: 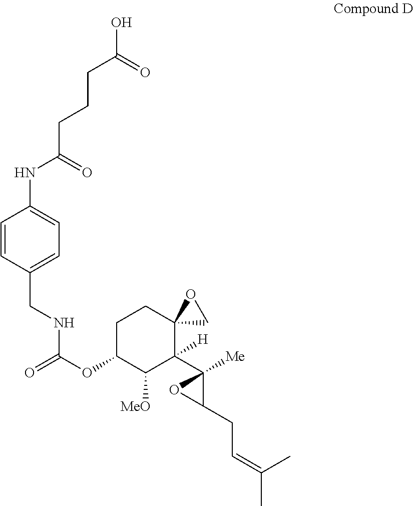
to a subject comprising administering to the subject a mixture of claim 1.
